(19) United States
(12) Patent Application Publication
(10) Pub. No.: US 2001/0038823 A1
(43) Pub. Date: Nov. 8, 2001

(54) MICROPARTICLES, PROCESS FOR THEIR PRODUCTION, AND THEIR USE IN ULTRASOUND DIAGNOSIS

(76) Inventors: GEORG ROSSLING, BERLIN (DE); CELAL ALBAYRAK, BERLIN (DE); JOHANNES TACK, BERLIN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/125,729

(22) PCT Filed: Mar. 11, 1997
(86) PCT No.: PCT/EP97/01185

§ 371 Date: None

(30) Foreign Application Priority Data
Mar. 14, 1996 (DE) 196 11 769.0

(51) Int. Cl. A61K 49/00

(52) U.S. Cl. 424/9.52

(57) ABSTRACT

The invention relates to gas-containing microparticles and nanoparticles that consist of biodegradable, synthetic polymers based on hydrophobized polysaccharides, agents that contain these particles for ultrasound diagnosis, and a process for the production of the particles and agents.

The invention relates to the subject that is characterized in the claims, i.e., gas-containing microparticles and nanoparticles that consist of biodegradable, synthetic polymers based on hydrophobized polysaccharides, agents that contain these particles for ultrasound diagnosis, and a process for the production of the particles and agents.

Because of its complication-free and simple handling, ultrasound diagnosis has become widely used in medicine. Ultrasonic waves are reflected at interfaces of media of different acoustic density. The echo signals that are produced in this case are electronically amplified and made visible.

The visualization of blood vessels and internal organs using ultrasound generally does not allow the visualization of blood flow. Liquids, especially blood, produce ultrasonic contrast only if density and compressibility differences exist with respect to the surrounding area. As contrast media, gas-containing or gas-producing substances are generally used in medical ultrasound diagnosis. In this respect, gases are especially suitable since here the impedance difference between the gas and surrounding blood is significantly greater than that observed for liquids or solids [Levine, R. A., J. Am. Coll. Cardiol. 3 (1988) 28 or Machi, I. J., CU 11 (1983) 3].

It is known that cardiac echo contrasts can be achieved with injections of solutions that contain fine gas bubbles (Roelandt, J., Ultrasound Med. Biol. 8 (1982) 471-492). These gas bubbles are produced in physiologically compatible solutions by, e.g., shaking or other agitation or by adding carbon dioxide. They are not standardized in terms of number and size, however, and cannot be adequately reproduced. Also, they are generally not stabilized, so that their life is short. Their mean diameters mostly exceed erythrocytes in size, so that they cannot pass through the pulmonary capillaries, with resultant contrasting of organs such as left heart, liver, kidney or spleen. Moreover, they are not suitable for quantification since the ultrasonic echo that they produce consists of several processes that cannot be separated from one another, such as bubble development, coalescence, and dissolution. Thus, it is not possible to obtain information on, e.g., transit times with the aid of these ultrasonic contrast media by measuring the path of the contrast in the myocardium.

For this purpose, contrast media are needed whose scatter elements exhibit adequate stability.

EP 0 131 540 describes the stabilization of gas bubbles with sugar. Thus, the reproducibility and homogeneity of the contrast effect are improved, but these bubbles do not survive passing through the lung.

EP 0 122 624 and 0 123 235 describe that the gas bubble-stabilizing effect of sugars, sugar alcohols, and salts is enhanced by adding surface-active substances. These contrast media offer passage through the pulmonary capillaries and the possibility of visualizing the arterial vascular space and various organs such as the liver or spleen. In this case, the contrast effect is limited to the vascular volume, however, since the bubbles are not taken up by the tissue cells.

None of the described ultrasonic contrast media remains unchanged in the body for a prolonged period of time. Organ visualization with sufficient signal intensity by selective concentration after i.v. administration or quantification is not possible with these media.

Encapsulation of gases such as, for example, air in particles and their use as ultrasonic contrast media are described in EP 0 224 934. The wall material that is used in this case consists of protein, especially human serum albumin with the known allergenic properties, to which cytotoxic effects can be added by denaturation.

Gas-containing microparticles for ultrasound diagnosis based on biodegradable, synthetic materials are described in European Patent Application EP 0 398 935. These media exhibit sufficient in vivo life, and after intravenous administration they are concentrated intracellularly in the reticulo-endothelial system and thus also in the liver or spleen.

European Patent Application EP 0 454 044 describes ultrasonic contrast media that are based on hydrophobized polysaccharides. In this case, only high-molecular, mixed polyelectrolyte complexes are used. Such complexes have a higher osmotic pressure in solution, however, than is measured for uncharged compounds; moreover, charged complexes generally exhibit worse in vivo compatibility than uncharged compounds.

The object of this invention was therefore to find ultrasonic contrast media that overcome the drawbacks of the prior art, i.e., to find contrast media that

provide a clear contrast with respect to the surrounding tissue,

that are small enough and stable enough that they reach the left side of the heart after intravenous administration without significant gas loss and basically quantitatively,

circulate optionally for a long time in the circulation,

have good compatibility without having allergenic potential,

do not aggregate together in water or blood and

can be produced quickly and easily.

The object is achieved by this invention.

It has been found that microparticles that consist of hydrophobized polysaccharides and a gas are extremely well suited for the production of a preparation for ultrasound diagnosis.

The gas-filled echogenic polymer nanoparticles or microparticles (also referred to below as particles or microparticles) according to the invention consist of biodegradable, synthetic polymers based on hydrophobized polysaccharides and have the advantage that they are easily degraded in vivo and without toxicologically harmful degradation products. Moreover, their lipophilic properties can be easily varied within wide ranges via the degree of esterification or etherification; this makes it possible to control the retention time in the circulation, as well as the dispersion behavior.

Since the wall thicknesses of the microparticles according to the invention can be influenced by the production process, particles can be produced whose oscillation modes can be stimulated by the sound field, thereby making it possible to use them even in nonlinear imaging modes.

The microparticles according to the invention are built up of hydrophobized polysaccharides. By way of example, there can be mentioned derivatives of hyaluronic acid, dextran, pullan, amylopectin, amylose, mannan and/or chitosan, whereby functional groups are completely or partially hydrophobized, i.e., esterified or etherified, by propyl, isopropyl, butyl, isobutyl, pentyl, isopentyl, hexyl, octyl, decyl, dodecyl, palmitoyl, stearinoyl, lauryl, and/or benzyl groups.

The degree of esterification or the degree of etherification (also referred to below generally as degree of substitution) is indicated in percent in this patent specification, whereby 100% esterification (esterification) is considered to be present if all functional groups (i.e., carboxyl or hydroxyl groups) of the polysaccharide are esterified (etherified). According to the invention, a degree of substitution of 30-100% is preferred.

The hydrophilia of the particles can be controlled by the degree of substitution and thus can influence the retention time in the blood. In general, the following is true: the more hydrophilic the particles, the longer the retention time in the circulation.

The more lipophilic particles also accumulate preferably in organs, such as, e.g., the liver, while particles with lower lipophilia preferably remain in the vascular space.

According to the invention, in addition polymers with a molecular weight of 10-250 kDalton are preferred.

Hyaluronic acid esters with the desired molecular weight and degree of esterification that can be used according to the invention can be produced according to the following processes that are known in the literature and are identified:

Jeanloz et al., Biol. Chem. 186, (1950) 495-511

Jeanloz et al., J. Biol. Chem. 194 (1952) 141-150

Jeanloz et al., Hel. Chimi. Act. 35 (1952) 262-271

Jager et al., J. Bacteriology. (1979) 1065-1067

U.S. Pat. No. 4,851,521

Kawaguchi et al., Carbohyd. Polym. 18 (1992) 139-141

Prestwich et al., Bioconjugate Chem. 5 (1994) 339-347

Prestwich et al., Bioconjugate Chem. 5 (1994) 370-372

Prestwich et al., Bioconjugate Chem. 2 (1991) 232-241

Chabrecek et al., J. Appl. Polym. Symp. 48 (1993) 20-22

Kobajashi et al., Biorheology 31 (1994) 235-244

Dextran, pullulan, amylopectin, amylose, mannan, chitosan, or chitin that is/are hydrophobized and can be used according to the invention can be produced according to the following processes that are known in the literature and are identified:

Suzuki, M. et al., Carbohydr. Res. 23 (223-229) 1977

Hammerling, U. et al., Biochimica et Biophysica Acta 875 9 (1986) 265-70

Kobojashi, K. et al., Makromolecules [Macromolecules] 19 (1986) 529-535

Ringsdorf, H. et al., Angew. Makromol. Chem. [Applied Macromol. Chem.] 166/167 (1989) 71-80

Sunamato, J. et al., CRC Critical Reviews in Therapeutic Drug Carrier Systems 2 (1986) 117-136

Ringsdorf, H. et al. Angew. Chem. Int. Ed. Engl. 27 (1988) 113

Toshihiro, S. et al., Makromol. Chem. 192 (1991) 2447-2461.

In addition to commonly used gases such as air, nitrogen, and noble gases, perfluorinated compounds are also suitable as gases that are contained in the particles.

Another subject of the invention is a process for the production of microparticles that consist of hydrophobized polysaccharides according to the invention.

For the production of microparticles, the procedure is such that the respective polymer and optionally a surface-active substance are dissolved in an organic solvent or solvent mixture. A perfluoro compound or water is dispersed in this solution. The dispersion is added to water, which optionally contains a surface-active substance, and is dispersed with the aid of a stirrer. The solvent is removed by introducing a gas (e.g., nitrogen) and optionally applying a vacuum. In this case, particles are formed that first also contain water or the liquid perfluoro compound. Then, the suspension that contains the particles is mixed with a suitable pharmaceutically acceptable cyroprotector and freeze-dried, whereby the liquid that is contained in the particles largely escapes and is replaced by the desired gas (generally air) after the freeze-drier is aerated. Depending on the drying time, optionally a small quantity of liquid (water or perfluoro compound) remains as vapor in the particles.

As perfluorinated liquid compounds, perfluoropentane, perfluorohexane, perfluoro-1,3-dimethylcyclohexane, perfluorocyclohexane, perfluorodecalin, and/or perfluoroether are used.

As organic solvents or solvent mixtures, dichloromethane, acetone, ethyl acetate, methyl acetate, triacetin, triethyl citrate, ethyl lactate, methyl lactate, propyl acetate, isopropyl acetate, propyl formate, butyl formate, and/or dimethyl sulfoxide are preferably used.

As a surface-active substance, preferably a substance from the group of Poloxamers®, Poloxamines®, polyethylene glycol alkyl ethers, polysorbates, saccharose esters (Sisterna®; The Netherlands), saccharose esters (Ryoto Sugarester®; Tokyo) and gelatin, polyvinyl alcohol, polyvinyl pyrrolidone, fatty alcohol polyglycoside, Chaps (Serva), Chap (Calbiochem), Chapso (Calbiochem), decyl-β-D-glycopyranoside, decyl-β-D-Dmaltopyranoside, sodium oleate, polyethylene glycol or mixtures thereof are used.

The production of the ready-to-use injectable preparations of the particles according to the invention is done by resuspending the lyophilizate in a pharmaceutically acceptable suspension medium. Suitable suspension media are, e.g., water p.i., aqueous solutions of one or more inorganic salts, such as, e.g., physiological electrolyte solutions or buffer solutions, such as, e.g., Tyrode's aqueous solutions of monosaccharides or disaccharides, such as glucose or lactose, sugar alcohols such as mannitol, which optionally in addition contain a surface-active substance from the group of polysorbates, polysaccharides, Poloxamers® or Poloxamines® and polyvinyl pyrrolidone, saccharose monoesters or diesters and/or a physiologically compatible multivalent alcohol, such as, e.g., glycerine. Water that is suitable for injection purposes is preferred, however.

To increase the reliability of the application, filtration of the suspension can be performed immediately before injection.

The agents according to the invention contain 10⁶-10¹⁰ particles per milliliter of suspension medium. The injected dose depends on the respective application; in ultrasonic-diagnostic studies of vessels, it is in the range of 1 to 500 μg, preferably between 10 and 100 μg of particles/kg of body weight, and in the study of the liver and spleen using color Doppler sonography, it is in the range of 50 to 1000, preferably between 200 and 600 μg/kg of body weight.

The microparticles according to the invention and the ultrasonic contrast media that are produced from them are distinguished by the following advantages:

They are quickly degraded in vivo,

Degradation products are toxicologically harmless,

They circulate for a sufficient time in the circulation, whereby the retention time can be controlled by the degree of substitution,

They can be used in all modes of ultrasound diagnosis, especially also in modes in which nonlinear effects are used,

They are well-tolerated,

They exhibit uniform, controllable size distributions,

They can be produced easily,

They are stable enough to survive passing through the lung and are thus also suitable for contrasting the left heart and

They are taken up from the reticuloendothelial system and are thus also suitable for contrasting the liver and spleen.

Moreover, the particles have excellent backscatter coefficients. The determination of the backscatter coefficient, which can be viewed as a yardstick of the effectiveness of the contrast media—was done in an in vitro test design, in which the “backscatter” that is produced by a contrast medium that is found in a vessel is measured (see “Standardization of the Measurement of Acoustical Parameters of Ultrasound Contrast Agents,” First European Symposium on Ultrasound Contrast Imaging, Jan. 25-26, 1996, Rotterdam).

The determination of particle size is done according to the Coulter-Counter method.

The following examples are used to explain the subject of the invention in more detail, without intending that it be limited to these examples.

### EXAMPLE 1

3.0 g of hyaluronic acid benzyl ester, in which all carboxyl groups are esterified (MW=160 kDal), is dissolved in 40 ml of methylene chloride. 10 ml of perfluoropentane is dispersed in the polymer solution using an Ultraturrax [10,000 rpm] for 2 minutes. The (O/O)-emulsion that is produced is dispersed in 400 ml of a 2% polyvinyl alcohol solution (PVA solution), which is temperature-equalized to 0° C., using a mechanical stirrer (Dispermat FT, VMA-Getzmann GmbH) for 30 minutes at 10,000 rpm. The (O/O/W) emulsion is moved in a three-necked flask that is equipped with a stirrer (300 rpm), and the solvent is removed for 3 hours at 20° C. by the introduction of N₂ and by vacuum. Then, the suspension is removed by ultrafiltration from the surfactant that is used and the residual solvent, in that the volume of the suspension is concentrated by evaporation to a minimum (50 ml) and the suspension is mixed with a pharmaceutically acceptable cryoprotector and freeze-dried.

The lyophilizate that is resuspended in water contains microparticles (diameter of 0.1-8 μm), and it has an excellent in vitro backscatter coefficient αₛ=2.5×10⁻¹ (dB/cm) at a 5 MHz transmission frequency and a particle concentration of c=4.0×10⁶ T/ml.

### EXAMPLE 2

The procedure is as in Example 1, whereby perfluoropentane is replaced by perfluorohexane. The lyophilizate that is resuspended in water contains ultrasound-active microparticles with a diameter of 0.1 to 8 μm.

### EXAMPLE 3

The procedure is as in Example (1), whereby the polymer hyaluronic acid benzyl ester that is used has a degree of esterification of 75%, and 40 ml of methylene chloride/ethyl acetate (volume proportion 2:1) is used as a solvent. The lyophilizate that is taken up in water contains ultrasound-active microparticles with a diameter of 0.1 to 8 μm.

### EXAMPLE 4

The procedure is as in Example (1), whereby the polymer hyaluronic acid benzyl ester is dissolved in 40 ml of methylene chloride/dimethyl sulfoxide (DMSO) (volume proportion 2:1). The particles that are resuspended in a 0.9% NaCl solution have an in vitro backscatter coefficient αₛ=2.3×10⁻¹ (dB/cm) at a 5 MHz transmission frequency and a particle concentration of c=3.6 ×10⁶ T/ml and have a diameter of 0.5 to 8 μm.

### EXAMPLE 5

The procedure is as in Example (1), whereby the polymer used is hyaluronic acid pentyl ester (degree of esterification 100%, MW=250 kDal), dissolved in 40 ml of methylene chloride/ethyl lactate (volume proportion 2:1). The lyophilizate that is taken up in a 5.5% mannitol solution contains ultrasound-active microparticles with a diameter of 0.1-6 μm.

### EXAMPLE 6

The procedure is as in Example (1), whereby the polymer used is hyaluronic acid palmitoyl ester (degree of esterification of 50%, MW=150 kDal). The lyophilizate that is resuspended in water contains ultrasound-active microparticles with a diameter of 0.3-8 μm.

### EXAMPLE 7

The procedure is as in Example (1), whereby the polymer hyaluronic acid benzyl ester is replaced by hyaluronic acid dodecyl ester (degree of esterification of 75%, MW=50 kDal). The lyophilizate that is resuspended in a 0.9% NaCl solution contains ultrasound-active microparticles with a diameter of 0.3-7 μm.

### EXAMPLE 8

3.0 g of palmitoyl dextran (degree of substitution 35%, MW=10-12 kDal) is dissolved in 40 ml of methylene chloride/isopropyl acetate (volume proportion 2:1). 10 ml of perfluoropentane is dispersed in the polymer solution using an Ultraturrax (10,000 rpm) for 2 minutes. The (O/O)-emulsion that is produced is dispersed in 400 ml of 2% PVA solution, which is temperature-equalized to 0° C. using a mechanical stirrer (Dispermat FT, VMA-Getzmann GmbH) for 30 minutes. The (O/O/W) emulsion is moved in a three-necked flask that is equipped with a stirrer (300 rpm), and is removed from solvent for 3 hours at 20° C. by introduction of N₂ and by vacuum. Then, the suspension is removed from the surfactant used and the residual solvent is removed by ultrafiltration, the volume of the suspension is concentrated by evaporation to a minimum (50 ml), and the suspension is mixed with a pharmaceutically acceptable cryoprotector and freeze-dried.

The lyophilizate that is resuspended in water contains microparticles with a diameter of 0.1 to 8 μm and have an excellent in vitro backscatter coefficient αₛ=2.0×10⁻¹ (dB/cm) at a 5 MHz transmission frequency and a particle concentration of c=4.0×10⁶ T/ml.

### EXAMPLE 9

The procedure is as in Example (8), whereby the polymer used is 3.0 g of succinic acid mono-N,N-bis(octadecyl)amine dextran (degree of substitution 20%; MW=8 kDal), and the solvent used is 40 ml of methylene chloride.

The lyophilizate that is resuspended in water contains ultrasound-active microparticles with a diameter of 0.1 to 6 μm.

### EXAMPLE 10

The procedure is as described in Example (8), whereby the polymer used is 3.0 g of palmitoylpullan (degree of substitution 30%; MW=51 kDal).

The lyophilizate that is resuspended in a 0.9% NaCl solution contains ultrasound-active microparticles with a diameter of 0.1 to 8 μm.

### EXAMPLE 11

The procedure is as described in Example (8), whereby the polymer used is 3.0 g of palmitoylamylopectin (degree of substitution 30%, MW=112 kdal) and the solvent used is 40 ml of methylene chloride. The lyophilizate that is resuspended in a 5.5% mannitol solution contains ultrasound-active microparticles with a diameter of 0.3 to 7 μm.

### EXAMPLE 12

The procedure is as in Example (11), whereby the polymer used is palmitoylamylose (degree of substitution 30%, MW=100 kDal) and the solvent used is 40 ml of methylene chloride/propyl formate (volume proportion 2:1).

The lyophilizate that is resuspended in water contains ultrasound-active microparticles with a diameter of 0.1 to 7 μm.

### EXAMPLE 13

The procedure is as in Example (11), whereby the polymer used is 3.0 g of palmitoylmannan with a degree of substitution of 35%.

The lyophilizate that is resuspended in water contains ultrasound-active microparticles with a diameter of 0.1 to 7 μm.

### EXAMPLE 14

The procedure is as in Example (11), whereby the polymer used is 3.0 g of palmitoylchitosan with a degree of substitution of 35%.

The lyophilizate that is resuspended in a 0.9% NaCl solution contains ultrasound-active microparticles with a diameter of 0.1 to 7 μm.

1. Microparticles for the production of a preparation for ultrasound diagnosis that consists of hydrophobized polysaccharides and a component that is in gaseous form at body temperature.

2. Microparticles according to claim 1, characterized in that as hydrophobized polysaccharides, derivatives of hyaluronic acid, dextran, pullan, amylopectin, amylose, mannan and/or chitosan are used, whereby functional groups are completely or partially esterified or etherified by propyl, isopropyl, butyl, isobutyl, pentyl, isopentyl, hexyl, octyl, decyl, dodecyl, palmitoyl, stearinoyl, lauryl, and/or benzyl groups.

3. Microparticles according to claim 1 or 2, wherein 30 to 100% of the functional groups of the polysaccharide are esterified or etherified.

4. Microparticles according to one of claims 1 to 3, wherein the hydrophobized polysaccharides have a molecular weight of 10-250 kdalton.

5. Microparticles according to one of claims 1 to 4, wherein the particles have a mean particle diameter of 500 nm to 10 μm.

6. Microparticles according to one of claims 1-5, wherein components air, nitrogen, and noble gases that are in gaseous form at body temperature are contained.

7. Microparticles according to one of claims 1-6, wherein perfluorinated compounds are contained as gaseous components.

8. Microparticles according to one of claims 7, wherein perfluoropentane, perfluorohexane, perfluoro-1,3dimethylcyclohexane, perfluorocyclohexane, perfluorodecalin, and/or perfluoroether is contained as a perfluorinated compound.

9. Microparticles according to one of claims 1-8, wherein hydrophobized polysaccharide, hyaluronic acid benzyl ester, hyaluronic acid pentyl ester, hyaluronic acid palmitoyl ester, hyaluronic acid dodecyl ester, palmitoyl dextran, succinic acid mono-N,N-bis(octadecyl)amine dextran, palmitoylpullan, palmitoylamylopectin, palmitoylamylose, palmitoylmannan, or palmitoylchitosan is used.

10. Ultrasonic contrast media that contain microparticles according to one of claims 1 to 9 in a physiologically compatible liquid suspension medium, optionally with the additives that are commonly used in pharmaceutical technology.

11. Ultrasonic contrast media according to claim 10 that contain as a physiologically compatible suspension medium water, aqueous solutions of one or more inorganic salts or aqueous solutions of monosaccharides or disaccharides, which optionally in addition contain a surface-active substance from the group of polysorbates, polysaccharides, Polxamers® or Poloxamines® as well as polyvinyl pyrrolidone, saccharose mono- or diesters and/or a physiologically compatible multivalent alcohol.

12. A kit for the production of an ultrasonic contrast medium that contains microparticles and gas that consists of
a) a first container, equipped with a closure, which makes it possible to remove the contents under sterile conditions and which is filled with liquid suspension medium, and
b) a second container, equipped with a closure, which makes it possible to add the suspension medium under sterile conditions, filled with microparticles according to one of claims 1 to 9 and a gas or gas mixture which is identical to the gas that is contained in the microparticles, whereby the volume of the second container is measured in such a way that there is enough room in the second container for the suspension medium of the first container.

13. Process for the production of a contrast medium that contains microparticles and gas for ultrasound diagnosis, wherein microparticles according to one of claims 1 to 9 are combined with a physiologically compatible carrier liquid and are shaken until a homogeneous suspension is produced.

14. Process for the production of microparticles according to one of claims 1 to 9, wherein the respective polymer and optionally a surface-active substance are dissolved in an organic solvent or solvent mixture, a perfluoro compound or water is dispersed in this solution, and then this dispersion is added to and dispersed in water, which optionally contains a surface-active substance, whereby the solvent or solvent mixture is removed by the introduction of gas and optionally application of a vacuum, and finally, the suspension that is thus obtained is mixed with a pharmaceutically acceptable cryoprotector and freeze-dried.
